(19) United States
(12) Patent Application Publication
(10) Pub. No.: US 2005/0119194 A1
(43) Pub. Date: Jun. 2, 2005

(54) Methods of using and compositions comprising thalidomide for treatment, modification and management of fibromyalgia

(76) Inventors: Jerome B. Zeldis, Princeton, NJ (US); Herbert Faleck, West Orange, NJ (US)

Correspondence Address: JONES DAY, 222 EAST 41ST ST, NEW YORK, NY, 10017, US

(21) Appl. No.: 10/972,311

(22) Filed: Oct. 22, 2004

Related U.S. Application Data
(60) Provisional application No. 60/514,548, filed on Oct. 24, 2003.

(51) Int. Cl. A61K 31/7048; A61K 31/454; A61K 31/553; A61K 31/55; A61K 31/485; A61K 31/445; A61K 31/506

(52) U.S. Cl. 514/28; 514/323; 514/165; 514/406; 514/217; 514/171; 514/211.13; 514/285; 514/389; 514/557; 514/252.15; 514/253.04; 514/570; 514/317; 514/355

(57) ABSTRACT

Methods of treating, preventing, modifying and/or managing fibromyalgia and related conditions are disclosed. Specific embodiments encompass the administration of thalidomide, or a pharmaceutically acceptable salt, solvate, hydrate, stereoisomer, clathrate, or prodrug thereof, alone or in combination with a second active agent and/or physical or psychological therapy. Pharmaceutical compositions, single unit dosage forms, and kits suitable for use in the methods of the invention are also disclosed.

This invention claims the benefit of U.S. Provisional Application No. 60/514,548, filed Oct. 24, 2003, which is incorporated herein in its entirety by reference.

### 1. FIELD OF THE INVENTION

This invention relates to methods of treating, preventing, modifying and/or managing fibromyalgia and related syndromes, which comprise the administration of thalidomide alone or in combination with known therapeutics. The invention also relates to pharmaceutical compositions and dosing regimens. In particular, the invention encompasses the use of thalidomide in conjunction with physical or psychological therapy and/or other standard therapies for fibromyalgia and related syndromes.

### 2. BACKGROUND OF THE INVENTION

Thalidomide is a racemic compound sold under the tradename Thalomid® and chemically named α-(N-phthalimido)glutarimide or 2-(2,6-dioxo-3-piperidinyl)-1H-isoindole-1,3(2H)-dione. The compound has structure I:

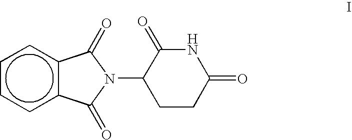

Thalidomide was originally developed in the 1950's to treat morning sickness, but due to its teratogenic effects was withdrawn from use. Thalidomide has been approved in the United States for the acute treatment of the cutaneous manifestations of erythema nodosum leprosum in leprosy. Physicians' Desk Reference, 1153-1157 (57th ed., 2003). Because its administration to pregnant women can cause birth defects, the sale of thalidomide is strictly controlled. Id. Thalidomide has been studied in the treatment of other diseases, such as chronic graft-vs-host disease, rheumatoid arthritis, sarcoidosis, several inflammatory skin diseases, and inflammatory bowel disease. See generally, Koch, H. P., Prog. Med. Chem. 22:165-242 (1985). See also, Moller, D. R., et al., J. Immunol. 159:5157-5161 (1997); Vasiliauskas, E. A., et al., Gastroenterology 117:1278-1287 (1999); Ehrenpreis, E. D., et al., Gastroenterology 117:1271-1277 (1999). It has further been alleged that thalidomide can be combined with other drugs to treat ischemia/repercussion associated with coronary and cerebral occlusion. See U.S. Pat. No. 5,643,915, which is incorporated herein by reference.

More recently, thalidomide was found to exert immunomodulatory and anti-inflammatory effects in a variety of disease states, cachexia in AIDS, and opportunistic infections in AIDS. In studies to define the physiological targets of thalidomide, the drug was found to have a wide variety of biological activities exclusive of its sedative effect including neurotoxicity, teratogenicity, suppression of TNF-α production by monocytes/macrophages and the accompanying inflammatory toxicities associated with high levels of TNF-α, and inhibition of angiogenesis and neovascularization.

Additionally, beneficial effects have been observed in a variety of dermatological conditions, ulcerative colitis, Crohn's disease, Bechets's syndrome, systemic lupus erythematosis, aphthous ulcers, cancer and lupus. The anti-angiogenic properties of thalidomide in in vivo models have been reported. D'Amato et al., Thalidomide Is An Inhibitor Of Angiogenesis, 1994, PNAS, USA 91:4082-4085.

However, a spectrum of activity of thalidomide is not fully characterized. Available data from in vitro studies and preliminary clinical trials suggest that the immunologic effects of the compound can vary substantially under different conditions. They may be related to suppression of tumor necrosis factor-alpha (TNF-α) production and downmodulation of selected cell surface adhesion molecules involved in leukocyte migration.

### 2.1 Pathobiology of Fibromyalgia

Fibromyalgia is a clinical syndrome of widespread pain, fatigue, poor sleep, and chronic aching in multiple areas of the musculoskeletal system characterized by reproducible increased tenderness at specific sites. Bradley et al., Rheum Dis Clin North Am 1999, 25(1): 56; and Pellegrino et al., Arch Phys Med Rehabil, 1999, 70: 61. Fibromyalgia is also known as FM, fibromyalgia syndrome (FMS), fibrositis, myofascial syndrome and widespread chronic pain syndrome. Pellegrino et al., Arch Phys Med Rehabil, 1999, 70: 61. Fibromyalgia may be secondary to trauma, rheumatic diseases such as rheumatoid arthritis and osteoarthritis, connective tissue disease, hypothyroidism, malignancy and other conditions. Id. When not associated with one of these conditions, the fibromyalgia is primary. Id.

Pain in patients with fibromyalgia diffusely radiates from the axial skeleton over large areas of the body, predominantly involving muscles, and is described as exhausting, burning, miserable, or unbearable. The 1990 American College of Rheumatology (ACR) classification criteria for the diagnosis of fibromyalgia are the presence of widespread pain for more than three months and pain, not just tenderness, that can be elicited by manual pressure of approximately 4 kg/cm2 at 11 or more defined tender points. Bradley et al., Rheum Dis Clin North Am 1999, 25(1): 56.

Pain in patients with fibromyalgia derives partly from a generalized decrease in the pain perception threshold, reflecting discrimination of a nociceptive quality from a nonnociceptive quality (e.g., touch, warmth, cold), and in the threshold for pain tolerance, reflecting an unwillingness to receive more intense stimulation. Bradley et al., Rheum Dis Clin North Am 1999, 25(1): 59.

Psychological distress plays a central role in the pain experience and overall morbidity of patients with fibromyalgia. Bradley et al., Rheum Dis Clin North Am 1999, 25(1): 59-60. Increased psychological distress is a common characteristic of fibromyalgia. Psychological distress strongly correlates with the number of tender points, both in patients with fibromyalgia and in the general population. High levels of anxiety and distress, less certainty of pain resolution, and a history of trauma are predictors of whether the patient will progress from acute pain to chronic pain. Id.

The incidence of the syndrome is higher in women than in men, although both sexes are affected. Pellegrino et al., Arch Phys Med Rehabil, 1999, 70: 62. The syndrome may occur in any age group, including the pediatric population. Gedalia et al., Clinical and Experimental Rheumatology, 2000, 18: 415-419. The cause of fibromyalgia appears to be multifactorial. Various causes include, but are not limited to, biological variables such as inheritance, abnormal sleep, trauma, tissue injury, malignancy, stress, neuroendocrine and autonomic dysregulation, neurotransmitter abnormalities, neuronal activation leading to central sensitization, low calcium levels, low serotonin levels, elevated levels of substance P, elevated levels of cerebrospinal fluid (CSF) nerve growth factor, elevated levels of CSF dynorphin A, elevated levels of CSF calcitonin gene-related peptide, and various other antinociceptive molecules, functional brain activity abnormalities (decreased regional blood flow in thalamus and caudate nucleus), neurally mediated hypotension, cognitive-behavioral variables such as adverse experiences during childhood, learned behaviors from living as children with dysfunctional or chronically ill parents, a failure in goal-oriented behavior leading to lower self-efficacy, the inability to achieve goals, a fear of failure, stress, depression and anxiety, and environmental and socio-cultural variables including psychosocial experiences during childhood and lack of spousal or family support. Bradley et al., Rheum Dis Clin North Am 1999, 25(1): 61-70; and Pellegrino et al., Arch Phys Med Rehabil, 1999, 70: 62.

### 2.2 Treatment of Fibromyalgia

Until now, few effective treatments exist for fibromyalgia and related syndromes. In some cases, physical therapy is used. Physical therapy for fibromyalgia includes massage and graded aerobic exercise such as low-impact aerobics, walking, water aerobics, swimming and stationary bicycle. Gedalia et al., Clinical and Experimental Rheumatology, 2000, 18: 418. However, excessive dependence on administration of physical therapy and modalities by another person may confound the patient's efforts to achieve self-efficacy for pain control.

Medications presently used for the treatment of fibromyalgia include anxiolytics, hypnotics, muscle relaxants, non-narcotic analgesics, opioid analgesics, antidepressants, anticonvulsants and antihypertensive agents. Gedalia et al., Clinical and Experimental Rheumatology, 2000, 18: 418. However, these drugs rarely yield complete pain relief. Therefore, there remains a need for safe and effective methods of treating and managing fibromyalgia.

### 3. SUMMARY OF THE INVENTION

This invention encompasses methods of treating or preventing fibromyalgia, which comprise administering to a patient in need thereof a therapeutically or prophylactically effective amount of thalidomide, or a pharmaceutically acceptable salt, solvate, hydrate, stereoisomer, clathrate, or prodrug thereof. The invention also encompasses methods of modifying or managing fibromyalgia, which comprise administering to a patient in need of such management a therapeutically or prophylactically effective amount of thalidomide, or a pharmaceutically acceptable salt, solvate, hydrate, stereoisomer, clathrate, or prodrug thereof.

Another embodiment of the invention encompasses the use of thalidomide in combination with other therapeutics presently used to treat or prevent fibromyalgia such as, but not limited to, antidepressants, antihypertensives, anxiolytics, hypnotics, anticonvulsants, calcium channel blockers, muscle relaxants, non-narcotic analgesics, opioid analgesics, alpha-adrenergic receptor agonists or antagonists, anti-inflammatory agents, cox-2 inhibitors, immunomodulatory agents, immunosuppressive agents, hyperbaric oxygen, JNK inhibitors and corticosteroids.

Yet another embodiment of the invention encompasses the use of thalidomide in combination with conventional therapies used to treat, prevent, modify or manage fibromyalgia including, but not limited to, surgery, interventional procedures (e.g., neural blockade), physical therapy, and psychological therapy.

The invention further encompasses pharmaceutical compositions, single unit dosage forms, and kits suitable for use in treating, preventing, modifying and/or managing fibromyalgia, which comprise thalidomide, or a pharmaceutically acceptable salt, solvate, hydrate, stereoisomer, clathrate, or prodrug thereof.

### 4. DETAILED DESCRIPTION OF THE INVENTION

This invention is based, in part, on the belief that compounds of the invention can work alone or in combination with other drugs to effectively treat, prevent, modify and/or manage varying types and severities of fibromyalgia. Without being limited by theory, compounds of the invention can, but do not necessarily, act as analgesics. In particular, because the compounds can dramatically affect the production of cytokines (e.g., TNF-α), it is believed that they can function as “antihyperalgesics” and/or “neuromodulators” by restoring the baseline or normal pain threshold of the injured animal of human to which they are administered. Thus, compounds of the invention can act differently than analgesics, which typically diminish the response induced by stimulus, by instead altering the patient's ability to withstand that response either by suppressing the suffering associated with the pain or directly reducing the responsiveness of the nociceptors. Moreover, because of the unique mechanism by which the compounds of the invention are believed to act, it is believed that they can relieve or reduce pain without incurring adverse effects (e.g., narcotic effects) typical of some analgesics (e.g., opioids), even when administered systemically.

A first embodiment of the invention encompasses methods of treating, preventing, modifying or managing fibromyalgia, which comprise administering to a patient in need thereof a therapeutically or prophylactically effective amount of thalidomide, or a pharmaceutically acceptable salt, solvate, hydrate, stereoisomer, clathrate, or prodrug thereof.

As used herein, the terms “fibromyalgia,” “fibromyalgia syndrome,” “myofascial syndrome” and “fibromyalgia and related syndromes” mean a chronic pain disorder characterized by one or more of the following: pain including allodynia (painful response to a stimulus that is not usually painful) and hyperalgesia (exaggerated response to a stimulus that is usually only mildly painful); a series of regional pains, such as noncardiac chest pain, dyspepsia, headache, abdominal cramping (irritable bowel syndrome), temporomandibular pain and chronic pelvic pain; stiffness; chronic aching in multiple areas of the musculoskeletal system; fatigue; poor sleep; tender points; cognitive difficulties with attention and memory; weight fluctuations; allergic symptoms (e.g., nasal congestion); hypersensitivity to environmental stimuli (e.g., odors, bright lights, loud noises) and medications; syncope; shortness of breath; and urinary frequency and urgency.

Another embodiment of the invention encompasses methods of modifying or modulating the threshold, development and duration of pain which comprise administering to a patient in need of such modification or modulation a therapeutically or prophylactically effective amount of thalidomide, or a pharmaceutically acceptable salt, solvate, hydrate, stereoisomer, clathrate, or prodrug thereof.

Another embodiment of the invention encompasses a pharmaceutical composition suitable for treatment, prevention, modification or management of fibromyalgia comprising thalidomide, or a pharmaceutically acceptable salt, solvate, hydrate, stereoisomer, clathrate, or prodrug thereof, and an optional carrier.

Also encompassed by the invention are single unit dosage forms suitable for use in treating, preventing, modifying or managing fibromyalgia comprising thalidomide, or a pharmaceutically acceptable salt, solvate, hydrate, stereoisomer, clathrate, or prodrug thereof, and an optional carrier.

Another embodiment of the invention encompasses a kit suitable for use in treating, preventing, modifying or managing fibromyalgia comprising: a pharmaceutical composition comprising thalidomide, or a pharmaceutically acceptable salt, solvate, hydrate, stereoisomer, clathrate, or prodrug thereof. The invention further encompasses kits comprising single unit dosage forms.

Without being limited by theory, it is believed that thalidomide and other medications used to treat symptoms of fibromyalgia can act in complementary or synergistic ways in the treatment, prevention, modification or management of fibromyalgia. Therefore, one embodiment of the invention encompasses a method of treating, preventing, modifying and/or managing fibromyalgia, which comprises administering to a patient in need thereof a therapeutically or prophylactically effective amount of thalidomide, or a pharmaceutically acceptable salt, solvate, hydrate, stereoisomer, clathrate, or prodrug thereof, and a therapeutically or prophylactically effective amount of a second active agent.

Examples of second active agents include, but are not limited to, antidepressants, antihypertensives, anxiolytics, hypnotics, anticonvulsants, calcium channel blockers, muscle relaxants, non-narcotic analgesics, opioid analgesics, alpha-adrenergic receptor agonists or antagonists, anti-inflammatory agents, cox-2 inhibitors, immunomodulatory agents, immunosuppressive agents, hyperbaric oxygen, JNK inhibitors, corticosteroids and other therapeutics found, for example, in the Physician's Desk Reference (2003).

The invention also encompasses pharmaceutical compositions, single unit dosage forms, and kits which comprise thalidomide, or a pharmaceutically acceptable salt, solvate, hydrate, stereoisomer, clathrate, or prodrug thereof, and a second active agent. For example, a kit may contain thalidomide and antidepressant, antihypertensive, anxiolytic, hypnotic, anticonvulsant, calcium channel blocker, muscle relaxant, non-narcotic analgesic, opioid analgesic, alpha-adrenergic receptor agonist or antagonist, anti-inflammatory agent, cox-2 inhibitor, immunomodulatory agent, immunosuppressive agent, hyperbaric oxygen, JNK inhibitor, corticosteroid or other drug capable of relieving or alleviating a symptom of fibromyalgia.

It is further believed that thalidomide can reduce or eliminate adverse effects associated with the administration of therapeutic agents used to treat fibromyalgia, thereby allowing the administration of larger amounts of those agents to patients and/or increasing patient compliance. Consequently, another embodiment of the invention encompasses a method of reversing, reducing or avoiding an adverse effect associated with the administration of a second active agent in a patient suffering from fibromyalgia, which comprises administering to a patient in need thereof a therapeutically or prophylactically effective amount of thalidomide, or a pharmaceutically acceptable salt, solvate, hydrate, stereoisomer, clathrate, or prodrug thereof. Examples of such adverse effects include, but are not limited to, emesis, insomnia and diarrhea.

As discussed elsewhere herein, symptoms of fibromyalgia may be treated with physical therapy and psychological therapy. Without being limited by theory, it is believed that the combined use of such conventional therapies and thalidomide can provide a uniquely effective treatment of fibromyalgia. Therefore, this invention encompasses a method of treating, preventing, modifying and/or managing fibromyalgia, which comprises administering to a patient (e.g., a human) thalidomide, or a pharmaceutically acceptable salt, solvate, hydrate, stereoisomer, clathrate, or prodrug thereof, before, during, or after physical therapy, psychological therapy or other conventional, non-drug based fibromyalgia therapies.

Compounds used in the invention include racemic thalidomide, stereomerically enriched or stereomerically pure thalidomide, and pharmaceutically acceptable salts, solvates, hydrates, stereoisomers, clathrates, and prodrugs thereof.

As used herein and unless otherwise indicated, the term “stereomerically pure” means a composition that comprises one stereoisomer of a compound and is substantially free of other stereoisomer of that compound. A typical stereomerically pure compound comprises greater than about 80% by weight of one stereoisomer of the compound and less than about 20% by weight of other stereoisomer of the compound, more preferably greater than about 90% by weight of one stereoisomer of the compound and less than about 10% by weight of the other stereoisomer of the compound, even more preferably greater than about 95% by weight of one stereoisomer of the compound and less than about 5% by weight of the other stereoisomer of the compound, and most preferably greater than about 97% by weight of one stereoisomer of the compound and less than about 3% by weight of the other stereoisomer of the compound.

As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of the compound.

As used herein and unless otherwise indicated, the term “enantiomerically pure” means a stereomerically pure composition of a compound having one chiral center. Similarly, the term “enantiomerically enriched” means a stereomerically enriched composition of a compound having one chiral center.

Thalidomide can either be commercially purchased (from Celgene Corp., New Jersey) or prepared according to the known methods. See, e.g., The Merck Index, p. 9182 (11^{th} ed.; 1989), and the references disclosed therein. Enantiomerically pure thalidomide can be resolved using known resolving agents or chiral columns as well as other standard synthetic organic chemistry techniques. See, e.g., Blaschke, Arzneimittelforschung 29: 1640-1642 (1979); Shealy et al., Chem. Indus. 1030 (1965); and Casini et al., Farmaco Ed. Sci. 19:563 (1964).

As used herein, unless otherwise specified, the term “pharmaceutically acceptable salt(s),” as used herein includes, but is not limited to, salts of acidic or basic moieties of thalidomide. Basic moieties are capable of forming a wide variety of salts with various inorganic and organic acids. The acids that can be used to prepare pharmaceutically acceptable acid addition salts of such basic compounds are those that form non-toxic acid addition salts, ie., salts containing pharmacologically acceptable anions. Suitable organic acids include, but are not limited to, maleic, fumaric, benzoic, ascorbic, succinic, acetic, formic, oxalic, propionic, tartaric, salicylic, citric, gluconic, lactic, mandelic, cinnamic, oleic, tannic, aspartic, stearic, palmitic, glycolic, glutamic, gluconic, glucaronic, saccharic, isonicotinic, methanesulfonic, ethanesulfonic, p-toluenesulfonic, benzenesulfonic acids, or pamoic (ie., 1,1′-methylene-bis-(2-hydroxy-3-naphthoate) acids. Suitable inorganic acids include, but are not limited to, hydrochloric, hydrobromic, hydroiodic, sulfuric, phosphoric, or nitric acids. Compounds that include an amine moiety can form pharmaceutically acceptable salts with various amino acids, in addition to the acids mentioned above.

Compounds that are acidic in nature are capable of forming salts with various pharmaceutically acceptable bases. The bases that can be used to prepare pharmaceutically acceptable base addition salts of such acidic compounds are those that form non-toxic base addition salts, ie., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of thalidomide that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of thalidomide that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5^{th} ed. 1995), and Design of Prodrugs (H. Bundgaard ed., Elselvier, N.Y. 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyloxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, a-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

### 4.1 Second Active Agents

A second active ingredient or agent can be used in the methods and compositions of the invention together with thalidomide. In a preferred embodiment, the second active agents are capable of relieving pain, inhibiting inflammatory reactions, providing a sedative effect or an antineuralgic effect, ameliorating depression and anxiety, or ensuring patient comfort.

Examples of the second active agents include, but are not limited to, opioid analgesics, non-narcotic analgesics, anti-inflammatories, cox-2 inhibitors, alpha-adrenergic receptor agonists or antagonists, ketamine, anesthetic agents, NMDA antagonists, immunomodulatory agents, immunosuppressive agents, antidepressants, hypnotics, anticonvulsants, antihypertensives, anxiolytics, calcium channel blockers, muscle relaxants, corticosteroids, hyperbaric oxygen, JNK inhibitors, other therapeutics known to relieve pain, and pharmaceutically acceptable salts, solvates, hydrates, stereoisomers, clathrates, prodrugs and pharmacologically active metabolites thereof.

Opioids can be used to treat severe pain. Examples of opioid analgesics include, but are not limited to, oxycodone (OxyContin®), morphine sulfate (MS Contin®, Duramorph®, Astramorph®), meperidine (Demerol®), and fentanyl transdermal patch (Duragesic®) and other known conventional medications; See, e.g., Physicians' Desk Reference, 2834, 2851 and 2991 (57th ed., 2003). Oxycodone (OxyContin®) is a long-acting form of an opioid and may be used usually in patients with fibromyalgia. Morphine sulfate may be used for analgesia due to reliable and predictable effects, safety profile, and ease of reversibility with naloxone. See, e.g., Physicians'Desk Reference, 2834 (57th ed., 2003).

Antihypertensive agents may be used in controlling withdrawal symptoms during tapering of opioids. Clonidine (Catapres®) stimulates alpha-2 adrenoreceptors in brain stem, activating an inhibitory neuron, which, in turn, results in reduced sympathetic outflow. See, e.g., Physicians' Desk Reference, 1033 (57th ed., 2003).

Non-narcotic analgesics and anti-inflammatories are preferably used for treatment of pain during pregnancy and breastfeeding. Anti-inflammatories such as non-steroidal anti-inflammatory drugs (NSAIDs) and cox-2 inhibitors typically inhibit inflammatory reactions and pain by decreasing the activity of cyclo-oxygenase, which is responsible for prostaglandin synthesis. NSAIDs may provide pain relief in the early stage of fibromyalgia. Examples of anti-inflammatories include, but are not limited to, salicylic acid acetate (Aspirin®), acetaminophen (Tylenol®, Feverall®, Tempra®, Aspirin-Free Anacin®), ibuprofen (Motrin®, Advil®), ketoprofen (Oruvail®), rofecoxib (Vioxx®), naproxen sodium (Anaprox®, Naprelan®, Naprosyn®), ketorolac (Acular®), and other known conventional medications. Specific cox-2 inhibitor is celecoxib (Celebrex®). See, e.g., Physicians'Desk Reference, 1065, 1903, 1910 and 2891 (57th ed., 2003); Physicians' Desk Reference for Nonprescription Drugs and Dietary Supplements, 511, 667 and 773 (23^{rd} ed., 2002).

Antidepressants increase the synaptic concentration of serotonin and/or norepinephrine in the CNS by inhibiting their reuptake by presynaptic neuronal membrane. Examples of antidepressants include, but are not limited to ketamine, nortriptyline (Pamelor®), amitriptyline (Elavil®), imipramine (Tofranil®), doxepin (Sinequan®), clomipramine (Anafranil®), fluoxetine (Prozac®), sertraline (Zoloft®), nefazodone (Serzone®), venlafaxine (Effexor®), trazodone (Desyrel®), bupropion (Wellbutrin®) and other known conventional medications. See, e.g., Physicians' Desk Reference, 329, 1417, 1831 and 3270 (57th ed., 2003).

Anticonvulsant drugs may also be used in embodiments of the invention for chronic pain states of fibromyalgia and related syndromes. They also may ameliorate depression and anxiety associated with pain. Examples of anticonvulsants include, but are not limited to, carbamazepine (Tegretol®), oxcarbazepine (Trileptal®), gabapentin (Neurontin®), phenytoin, sodium valproate, clonazepam, topiramate, lamotrigine, zonisamide, and tiagabine. See, e.g., Physicians' Desk Reference, 2323 (57th ed., 2003).

Anxiolytics and hypnotics can be used for relieving anxiety and panic, and as sleep aids because poor sleep is nearly universal in fibromyalgia. They also have antinociceptive effects in patients with chronic pain. Anxiolytics sometimes are used in combination with antidepressants and antiepileptic drugs. Alprazolam (Xanax®), lorazepam (Ativan®), clonazepam (Klonopin®), buspirone (BuSpar®), trazodone (Desyrel®), zolpidem (Ambien®) and temazepam (Restoril®) are useful in combination with thalidomide. See, e.g., Physicians' Desk Reference, 1280, 2517 and 2794 (57th ed., 2003).

Skeletal muscle relaxants (e.g., cyclobenzaprine (Flexeril®) or carisoprodol (Soma®)) can be used as adjunctive therapy for nociceptive pain associated with muscle strains. See, e.g., Physicians' Desk Reference, 1897 and 3254 (57th ed., 2003).

Corticosteroids (e.g., prednisone, dexamethasone or hydrocortisone), orally active class lb anti-arrhythmic agents (e.g., mexiletine), calcium channel blockers (e.g., nifedipine), beta-blockers (e.g., propranolol), alpha-blocker (e.g., phenoxybenzamine), and alpha2-adrenergic agonists (e.g., clonidine) can also be used in combination with thalidomide. See, e.g., Physicians' Desk Reference, 1034, 1979, 2190 and 3404 (57th ed., 2003).

Specific second active agents used in the invention include, but are not limited to, salicylic acid acetate (Aspirin®), acetaminophen (Tylenol), celecoxib (Celebrex®), Enbrel®, ketamine, gabapentin (Neurontin®), phenytoin (Dilantin®), carbamazepine (Tegretol®), oxcarbazepine (Trileptal®), valproic acid (Depakene®), morphine sulfate, hydromorphone, prednisone, griseofulvin, penthonium, alendronate, dyphenhydramide, guanethidine, ketorolac (Acular®), thyrocalcitonin, dimethylsulfoxide (DMSO), clonidine (Catapress®), bretylium, ketanserin, reserpine, droperidol, atropine, phentolamine, bupivacaine, lidocaine, acetaminophen, nortriptyline (Pamelor®), amitriptyline (Elavil®), imipramine (Tofranil®), doxepin (Sinequan®), clomipramine (Anafranil®), fluoxetine (Prozac®), sertraline (Zoloft®), nefazodone (Serzone®), venlafaxine (Effexor®), trazodone (Desyrel®), bupropion (Wellbutrin®), mexiletine, nifedipine, propranolol, tramadol, lamotrigine, ziconotide, ketamine, dextromethorphan, benzodiazepines, baclofen, tizanidine, phenoxybenzamine, alprazolam (Xanax®), lorazepam (Ativan®), clonazepam (Klonopin®), buspirone (BuSpar®), trazodone (Desyrel®), zolpidem (Ambien®), temazepam (Restoril®), cyclobenzaprine (Flexeril®) and carisoprodol (Soma®).

### 4.2 Methods of Treatment and Management

Methods of this invention encompass methods of preventing, treating, modifying and/or managing various types of fibromyalgia and related syndromes. As used herein, unless otherwise specified, the term “preventing fibromyalgia” includes, but is not limited to, inhibiting or reducing the severity of one or more symptoms associated with fibromyalgia. Symptoms associated with fibromyalgia include, but are not limited to, pain including allodynia (painful response to a stimulus that is not usually painful) and hyperalgesia (exaggerated response to a stimulus that is usually only mildly painful); a series of regional pains, such as noncardiac chest pain, dyspepsia, headache, abdominal cramping (irritable bowel syndrome), temporomandibular pain and chronic pelvic pain; fatigue; poor sleep; tender points; cognitive difficulties with attention and memory; weight fluctuations; allergic symptoms (e.g., nasal congestion); hypersensitivity to environmental stimuli (e.g., odors, bright lights, loud noises) and medications; syncope; shortness of breath; urinary frequency and urgency; autonomic dysfunction; depression; and anxiety.

As used herein, unless otherwise specified, the term “treating fibromyalgia” refers to the administration of thalidomide or other additional active agent after the onset of symptoms of fibromyalgia whereas “preventing” refers to the administration prior to the onset of symptoms, particularly to patients at risk of fibromyalgia. Examples of patients at risk of fibromyalgia include, but are not limited to, those who have incidents of trauma, neurologic disorder, myocardial infarction, musculoskeletal disorder, rheumatic disease, connective tissue disease, hypothyroidism and malignancy. Patients with familial history of fibromyalgia and related syndromes are also preferred candidates for preventive regimens.

As used herein and unless otherwise indicated, the term “modifying fibromyalgia” encompasses modulating the threshold, development and/or duration of pain, or changing the way that a patient responds to pain. Without being limited by theory, it is believed that compounds of the invention can act as an antihyperalgesic and/or neuromodulator. In one embodiment, “modifying fibromyalgia” encompasses reducing or eliminating an exaggerated pain response of a patient (i.e., a level at which a patient experiences greater than normal pain in response to a particular stimulus), taking the system of a human or animal back towards a normal pain threshold. In another embodiment, “modifying fibromyalgia” encompasses reducing a patient's pain response to a stimulus of a particular intensity. In another embodiment, “modifying fibromyalgia” encompasses increasing a patient's pain threshold relative to the patient's pain threshold prior to such modification.

As used herein and unless otherwise indicated, the term “managing fibromyalgia” encompasses preventing the recurrence of fibromyalgia in a patient who had suffered from fibromyalgia, and/or lengthening the time that a patient who had suffered from fibromyalgia remains in remission.

The invention encompasses methods of treating, preventing, modifying and managing fibromyalgia and related syndromes in patients with various stages and specific types of the disease, including, but not limited to, those referred to as FM, fibromyalgia syndrome (FMS), fibrositis, myofascial syndrome, widespread chronic pain syndrome, chronic fatigue syndrome, radiculopathy, and other painful neuropathic conditions, e.g., diabetic neuropathy. It further encompasses methods of treating patients who have been previously treated for fibromyalgia but non-responsive to standard therapy, as well as those who have not previously been treated for fibromyalgia. Because patients with fibromyalgia have heterogenous clinical manifestations and varying clinical outcomes, the treatment given to a patient may vary, depending on his/her prognosis. The skilled clinician will be able to readily determine without undue experimentation specific secondary agents and types of physical therapy that can be effectively used to treat an individual patient.

Methods encompassed by this invention comprise administering thalidomide, or a pharmaceutically acceptable salt, solvate, hydrate, stereoisomer, clathrate, or prodrug thereof to a patient (e.g., a human) suffering, or likely to suffer, from fibromyalgia.

In one embodiment of the invention, thalidomide is administered orally and in single or divided daily doses in an amount of from about 50 to about 1,500 mg/day, preferably from about 50 to about 1,000 mg/day, and more preferably from about 100 to about 400 mg/day (e.g., about 300 mg/day). In one embodiment, thalidomide is administered orally and in single or divided daily doses in an amount of from about 100 to about 400 mg/day to patients with fibromyalgia. In another embodiment, thalidomide is administered every other day, several times per week, or in another syncopated regimen.

In another embodiment, the invention relates to a method of treating, preventing, managing and/or modifying fibromyalgia associated with planned surgery (i.e., planned trauma), comprising administering an effective amount of thalidomide to a patient in need thereof. In this embodiment, thalidomide can be administered before, during and/or after the planned surgery. In an embodiment, the patient is administered with about 1 to about 200 mg/day, about 1 to about 100 mg/day, about 1 to about 75 mg/day, about 1 to about 50 mg/day, about 5 to about 40 mg/day, about 5 to about 25 mg/day, or about 5 to about 10 mg/day, of thalidomide from about 1-21 days prior to the planned surgery and/or from about 1-21 days after the planned surgery. In another embodiment, the patient is administered with about 10 mg/day of thalidomide from about 1-21 days prior to the planned surgery and/or from about 1-21 days after the planned surgery.

### 4.2.1 Combination Therapy with a Second Active Agent

Specific methods of the invention comprise administering thalidomide, or a pharmaceutically acceptable salt, solvate, hydrate, stereoisomer, clathrate, or prodrug thereof, in combination with a second active agent or active ingredient. Examples of second active agents are also disclosed herein (see, e.g., section 4.1).

Administration of thalidomide and the second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration for thalidomide is oral. Preferred routes of administration for the second active agents or ingredients of the invention are known to those of ordinary skill in the art. See, e.g., Physicians'Desk Reference, 594-597 (57th ed., 2003).

In one embodiment, the second active agent is administered orally, intravenously, intramuscularly, subcutaneously, mucosally, or transdermally and once or twice daily in an amount of from about 1 to about 3,500 mg, from about 5 to about 2,500 mg, from about 10 to about 500 mg, or from about 25 to about 250 mg.

The specific amount of the second active agent will depend on the specific agent used, the type of fibromyalgia being treated or managed, the severity and stage of fibromyalgia, and the amount(s) of thalidomide and any optional additional active agents concurrently administered to the patient. In a particular embodiment, the second active agent is IMiDs™, SelCIDs™, salicylic acid acetate (Aspirin®), acetaminophen (Tylenol), celecoxib (Celebrex®), Enbrel®, ketamine, gabapentin (Neurontin®), phenytoin (Dilantin®), carbamazepine (Tegretol®), oxcarbazepine (Trileptal®), valproic acid (Depakene®), morphine sulfate, hydromorphone, prednisone, griseofulvin, penthonium, alendronate, dyphenhydramide, guanethidine, ketorolac (Acular®), thyrocalcitonin, dimethylsulfoxide (DMSO), clonidine (Catapress®), bretylium, ketanserin, reserpine, droperidol, atropine, phentolamine, bupivacaine, lidocaine, acetaminophen, nortriptyline (Pamelor®), amitriptyline (Elavil®), imipramine (Tofranil®), doxepin (Sinequan®), clomipramine (Anafranil®), fluoxetine (Prozac®), sertraline (Zoloft®), nefazodone (Serzone®), venlafaxine (Effexor®), trazodone (Desyrel®), bupropion (Wellbutrin®), mexiletine, nifedipine, propranolol, tramadol, lamotrigine, ziconotide, ketamine, dextromethorphan, benzodiazepines, baclofen, tizanidine, phenoxybenzamine, alprazolam (Xanax®), lorazepam (Ativan®), clonazepam (Klonopin®), buspirone (BuSpar®), trazodone (Desyrel®), zolpidem (Ambien®), temazepam (Restoril®), cyclobenzaprine (Flexeril®), carisoprodol (Soma®) or a combination thereof, or a pharmaceutically acceptable salt, solvate, hydrate, stereoisomer, clathrate, prodrug or pharmacologically active metabolite thereof.

Hydromorphone (Dilaudid®) is preferably administered in an initial dose of about 2 mg orally, or about 1 mg intravenously to manage moderate to severe pain. See, e.g., Physicians' Desk Reference, 441-446 (57th ed., 2003). Morphine sulphate (Duramorph®, Astramorph®, MS Contin®) is preferably administered in an initial dose of about 2 mg IV/SC/IM, depending on whether a patient has already taken narcotic analgesics. See, e.g., Physicians' Desk Reference, 594-595 (57th ed., 2003). Morphine sulphate are also available in oral form in immediate-release and timed-release preparations. The long-acting oral form may be administered twice per day. An immediate-release form may be needed for periods of pain break-through, with the dose dependent on previous use. Oxycodone (OxyContin®) is a long-acting form of an opioid and may be used in initial and later stages of fibromyalgia. Oxycodone (OxyContin®) is preferably administered in an amount of about 10-160 mg twice a day. See, e.g., Physicians' Desk Reference, 2851 (57th ed., 2003). Meperidine (Demerol®) is preferably administered in an amount of about 50-150 mg PO/IV/IM/SC every 3-4 hours. A typical pediatric dose of meperidine (Demerol®) is 1-1.8 mg/kg (0.5-0.8 mg/lb) PO/IV/IM/SC every 3-4 hours. See, e.g., Physicians' Desk Reference, 2991 (57th ed., 2003).

Non-narcotic analgesics and anti-inflammatories such as NSAIDs and cox-2 inhibitors may be used to treat patients suffering from mild to moderate pain. Ibuprofen (Motrin®, Advil®) is orally administered in an amount of 400-800 mg three times a day. See, e.g., Physicians' Desk Reference, 1900 (57th ed., 2003); Physicians' Desk Reference for Nonprescription Drugs and Dietary Supplements, 511, 667, 773 (23^{rd} ed., 2002). Naproxen sodium (Anaprox®, Naprelan®, Naprosyn®) may also preferably be used for relief of mild to moderate pain in an amount of about 275 mg thrice a day or about 550 mg twice a day. See, e.g., Physicians' Desk Reference, 2891 (57th ed., 2003). Acetaminophen (Tylenol®, Feverall®, Tempra®, Aspirin-Free Anacin®) may preferably be used for pain in patients with hypersensitivity to aspirin or NSAIDs, in an amount of about 325-650 mg every four to six hours or about 1,000 mg three or four times a day. See, e.g., Physicians' Desk Reference, 1910-1914 (57th ed., 2003).

Antidepressants, e.g., nortriptyline (Pamelor®), may also be used in embodiments of the invention to treat patients suffering from chronic and/or neuropathic pain. The oral adult dose is typically in an amount of about 25-100 mg, and preferably does not exceed 200 mg/d. A typical pediatric dose is about 0.1 mg/kg PO as initial dose, increasing, as tolerated, up to about 0.5-2 mg/d. Amitriptyline (Etrafon®) is preferably used for neuropathic pain in an adult dose of about 10-100 mg PO. See, e.g., Physicians' Desk Reference, 329, 1417, 1831 and 3270 (57th ed., 2003).

Anticonvulsants such as gabapentin (Neurontin®) may also be used to treat patients suffering from chronic and neuropathic pain. Preferably, gabapentin is orally administered in an amount of about 100-1,200 mg three or four times a day. See, e.g., Physicians' Desk Reference, 2563 (57th ed., 2003). Carbamazepine (Tegretol®) is used to treat pain associated with true trigeminal neuralgia. The oral adult dose is typically in an amount of about 100 mg twice a day as initial dose, increasing, as tolerated, up to about 2,400 mg/d. See, e.g., Physicians' Desk Reference, 2323-25 (57th ed., 2003).

Anxiolytics and hypnotics such as alprazolam (Xanax®), lorazepam (Ativan®), clonazepam (Klonopin®), buspirone (BuSpar®), trazodone (Desyrel®), zolpidem (Ambien®) and temazepam (Restoril®) can be used for relieving pain, anxiety and panic, and as sleep aids in patients with fibromyalgia in an amount of about 0.25-300 mg a day in divided doses. See, e.g., Physicians' Desk Reference, 1280, 2517 and 2794 (57th ed., 2003).

Skeletal muscle relaxants can be used as adjunctive therapy for pain associated with muscle strains. Cyclobenzaprine (Flexeril®) is orally administered in an amount of about 20-40 mg a day in divided doses. Carisoprodol (Soma®) is orally administered in an amount of about 350 mg a day in divided doses. See, e.g., Physicians' Desk Reference, 1897 and 3254 (57th ed., 2003).

### 4.2.2 Use with Physical Therapy or Psychological Therapy

In still another embodiment, this invention encompasses a method of treating, preventing, modifying and/or managing fibromyalgia, which comprises administering thalidomide, or a pharmaceutically acceptable salt, solvate, hydrate, stereoisomer, clathrate, or prodrug thereof, in conjunction with physical therapy or psychological therapy.

The benefits of exercise for patients with fibromyalgia include improvement in subjective and objective measures of pain and in an overall sense of well-being. Because many patients with chronic pain fear that activity will make their pain and fatigue worse, they become deconditioned. Physical therapy (e.g., low-impact aerobics, walking, water aerobics, stationary bicycle, massage and swimming) can thus play an important role in functional restoration.

It is believed that the combined use of the thalidomide and physical therapy can provide a unique treatment regimen that is unexpectedly effective in certain patients.

It is believed that the combined use of thalidomide and psychological treatment can provide a unique treatment regimen that is unexpectedly effective in certain patients. Without being limited by theory, it is believed that thalidomide can provide additive or synergistic effects when given concurrently with psychological therapy including, but not limited to, biofeedback, relaxation training, activity pacing, visual imagery, distraction strategies, cognitive-behavioral therapy, and individual or family psychotherapy.

Thalidomide, or a pharmaceutically acceptable salt, solvate, hydrate, stereoisomer, clathrate, or prodrug thereof is administered before, during, or after physical therapy or psychological treatment. In specific methods, a second active agent is also administered to the patient.

### 4.3 Pharmaceutical Compositions and Single Unit Dosage Forms

Pharmaceutical compositions can be used in the preparation of individual, single unit dosage forms. Pharmaceutical compositions and dosage forms of the invention comprise thalidomide, or a pharmaceutically acceptable salt, solvate, hydrate, stereoisomer, clathrate, or prodrug thereof. Pharmaceutical compositions and dosage forms of the invention can further comprise one or more excipients.

Pharmaceutical compositions and dosage forms of the invention can also comprise one or more additional active ingredients. Consequently, pharmaceutical compositions and dosage forms of the invention comprise the active agents disclosed herein (e.g., thalidomide, or a pharmaceutically acceptable salt, solvate, hydrate, stereoisomer, clathrate, or prodrug thereof, and a second active agent). Examples of optional additional active agents are disclosed herein (see, e.g., section 4.1).

Single unit dosage forms of the invention are suitable for oral, mucosal (e.g., nasal, sublingual, vaginal, buccal, or rectal), or parenteral (e.g., subcutaneous, intravenous, bolus injection, intramuscular, or intraarterial), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18^{th} ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise thalidomide or a pharmaceutically acceptable salt, solvate, hydrate, stereoisomer, clathrate, or prodrug thereof in an amount of from about 50 to about 1,500 mg. Typical dosage forms comprise thalidomide or a pharmaceutically acceptable salt, solvate, hydrate, stereoisomer, clathrate, or prodrug thereof in an amount of about 50, 100, 200, 300 or 400 mg. Typical dosage forms comprise the second active ingredient in an amount of form about 1 to about 3,500 mg, from about 5 to about 2,500 mg, from about 10 to about 500 mg, or from about 25 to about 250 mg. Of course, the specific amount of the second active ingredient will depend on the specific agent used, the type of fibromyalgia being treated or managed, and the amount(s) of thalidomide and any optional additional active agents concurrently administered to the patient.

### 4.3.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18^{th} ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A preferred solid oral dosage form of the invention comprises thalidomide, anhydrous lactose, microcrystalline cellulose, polyvinylpyrrolidone, stearic acid, colloidal anhydrous silica, and gelatin. See, e.g., U.S. patent application Ser. No. 10/608,077 filed Jun. 30, 2003, the entirety of which is incorporated herein by reference.

### 4.3.2 Delayed Release Dosage Forms

Active agents of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 4.3.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention. For example, cyclodextrin and its derivatives can be used to increase the solubility of thalidomide. See, e.g., U.S. Pat. No. 5,134,127, which is incorporated herein by reference.

### 4.3.4 Topical and Mucosal Dosage Forms

Topical and mucosal dosage forms of the invention include, but are not limited to, sprays, aerosols, solutions, emulsions, suspensions, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4^{th} ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 4.3.5 Kits

Typically, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a dosage form of thalidomide, or a pharmaceutically acceptable salt, solvate, hydrate, stereoisomer, prodrug, or clathrate thereof. Kits encompassed by this invention can further comprise additional active agents or a combination thereof. Examples of the additional active agents include, but are not limited to, antidepressants, anticonvulsants, antihypertensives, anxiolytics, calcium channel blockers, muscle relaxants, non-narcotic analgesics, opioid analgesics, anti-inflammatories, cox-2 inhibitors, immunomodulatory agents, immunosuppressive agents, corticosteroids, or other therapeutics discussed herein (see, e.g., section 4.1).

Kits of the invention can further comprise devices that are used to administer the active agents. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 5. EXAMPLES

The following studies are intended to further illustrate the invention without limiting its scope.

### 5.1 Pharmacology Studies

One of biological effects exerted by thalidomide is the reduction of synthesis of TNF-α. Thalidomide enhances the degradation of TNF-α mRNA. TNF-α may play a pathological role in neuropathic pain. Increase of its expression in Schwann cells is shown in human painful neuropathies. Soluble TNF-α receptors are increased in the serum of patients with allodynia, as compared with neuropathy patients who do not report allodynia. The cytokine can induce ectopic activity in primary afferent nociceptors, and thus is a potential cause of pain and hyperalgesia in fibromyalgia. One possible mechanism of this is that TNF-α can form active sodium ion channels in cells. Increased influx of sodium into nociceptors would dispose them toward ectopic discharge. The cytokine may play a pathological role if it is active at sites of nerve damage or dysfunction.

Inhibition of TNF-α production following LPS-stimulation of human PBMC by thalidomide was investigated in vitro. The IC₅₀'s of thalidomide for inhibiting production of TNF-α following LPS-stimulation of PBMC was ˜194 μM (50.1 μg/nL).

### 5.2 Clinical Studies in Fibromyalgia Patients

Pressure of approximate 4 kg/cm² is applied manually at multiple anatomic points of patients with fibromyalgia to evaluate pain and tenderness. A useful device for quantitating pain sensitivity is a pressure algometer or dolorimeter. With this instrument or with multiple other laboratory approaches, such as the use of a thermode, a generalized decrease in the pain perception threshold and pain tolerance threshold has been demonstrated unequivocally in patients with fibromyalgia and related syndromes. This reflects altered central nociceptive processing and is expressed in the patient as allodynia and hyperalgesia.

Clinical studies are performed in fibromyalgia patients that have not responded to conventional physical therapy and have been present for at least one year. In one embodiment, thalidomide is administered in an amount of 100 to 400 mg per day to patients with fibromyalgia for from three months to one year. A baseline evaluation is performed for the effect of the drug treatment on pain intensity, impact of pain on activities of daily living, and consumption of other pain medications. Treatment with 300 mg as a continuous oral daily dose is well-tolerated. The study in fibromyalgia patients treated with thalidomide suggests that the drug has analgesic benefit in this disease.

Embodiments of the invention described herein are only a sampling of the scope of the invention. The full scope of the invention is better understood with reference to the attached claims. All of the references cited herein are incorporated herein in their entireties. None of the references cited herein are admitted to be prior art.

1. A method of treating, preventing, modifying or managing fibromyalgia, which comprises administering to a patient in need of such treatment, prevention, modification or management of a therapeutically or prophylactically effective amount of thalidomide, or a pharmaceutically acceptable salt, solvate, or stereoisomer thereof.

2. A method of treating, preventing, modifying or managing fibromyalgia, which comprises administering to a patient in need of such treatment, prevention, modification or management of a therapeutically or prophylactically effective amount of thalidomide, or a pharmaceutically acceptable salt, solvate, or stereoisomer thereof, and a therapeutically or prophylactically effective amount of a second active agent.

3. The method of claim 2, wherein the second active agent is capable of relieving pain, depression or anxiety.

4. The method of claim 2, wherein the second active agent is an antidepressant, anxiolytic, hypnotic, antihypertensive, anticonvulsant, calcium channel blocker, muscle relaxant, non-narcotic analgesic, opioid analgesic, alpha-adrenergic receptor agonist, alpha-adrenergic receptor antagonist, anti-inflammatory agent, cox-2 inhibitor, immunomodulatory agent, immunosuppressive agent, hyperbaric oxygen, JNK inhibitor, or corticosteroid.

5. The method of claim 2, wherein the second active agent is salicylic acid acetate, celecoxib, ketamine, gabapentin, carbamazepine, oxcarbazepine, phenytoin, sodium valproate, prednisone, nifedipine, clonidine, oxycodone, meperidine, morphine sulfate, hydromorphone, fentanyl, acetaminophen, ibuprofen, naproxen sodium, griseofulvin, amitriptyline, imipramine, doxepin, alprazolam, lorazepam, clonazepam, buspirone, trazodone, zolpidem, temazepam, cyclobenzaprine or carisoprodol.

6. The method of claim 1 or 2, wherein the fibromyalgia is fibrositis, myofascial syndrome, widespread chronic pain syndrome, chronic fatigue syndrome, radiculopathy, or painful neuropathic condition.

7. The method of claim 1 or 2, wherein the thalidomide is enantiomerically pure.

8. A method of treating, preventing, modifying or managing fibromyalgia, which comprises administering to a patient in need of such treatment, prevention, modification or management a therapeutically or prophylactically effective amount of thalidomide, or a pharmaceutically acceptable salt, solvate, or stereoisomer thereof, before, during or after physical therapy or psychological therapy directed at reducing or avoiding a symptom of fibromyalgia in the patient.

9. A method of reducing or avoiding an adverse effect associated with the administration of a second active ingredient in a patient suffering from fibromyalgia, which comprises administering to a patient in need of such reduction or avoidance an effective amount of thalidomide, or a pharmaceutically acceptable salt, solvate, or stereoisomer thereof.

10. The method of claim 9, wherein the second active agent is capable of relieving pain, depression or anxiety.

11. The method of claim 9, wherein the second active agent is an antidepressant, anxiolytic, hypnotic, antihypertensive, anticonvulsant, calcium channel blocker, muscle relaxant, non-narcotic analgesic, opioid analgesic, alpha-adrenergic receptor agonist, alpha-adrenergic receptor antagonist, anti-inflammatory agent, cox-2 inhibitor, immunomodulatory agent, immunosuppressive agent, hyperbaric oxygen, JNK inhibitor, or corticosteroid.

12. The method of claim 9, wherein the second active agent is salicylic acid acetate, celecoxib, ketamine, gabapentin, carbamazepine, oxcarbazepine, phenytoin, sodium valproate, prednisone, nifedipine, clonidine, oxycodone, meperidine, morphine sulfate, hydromorphone, fentanyl, acetaminophen, ibuprofen, naproxen sodium, griseofulvin, amitriptyline, imipramine, doxepin, alprazolam, lorazepam, clonazepam, buspirone, trazodone, zolpidem, temazepam, cyclobenzaprine or carisoprodol.

13. A pharmaceutical composition comprising thalidomide, or a pharmaceutically acceptable salt, solvate, or stereoisomer thereof, and a second active agent, wherein the second active agent is an antidepressant, anxiolytic, hypnotic, antihypertensive, anticonvulsant, calcium channel blocker, muscle relaxant, non-narcotic analgesic, opioid analgesic, alpha-adrenergic receptor agonist, alpha-adrenergic receptor antagonist, anti-inflammatory agent, cox-2 inhibitor, immunomodulatory agent, immunosuppressive agent, hyperbaric oxygen, JNK inhibitor, or corticosteroid.

14. The pharmaceutical composition of claim 13, wherein the second active ingredient is salicylic acid acetate, celecoxib, ketamine, gabapentin, carbamazepine, oxcarbazepine, phenytoin, sodium valproate, prednisone, nifedipine, clonidine, oxycodone, meperidine, morphine sulfate, hydromorphone, fentanyl, acetaminophen, ibuprofen, naproxen sodium, griseofulvin, amitriptyline, imipramine, doxepin, alprazolam, lorazepam, clonazepam, buspirone, trazodone, zolpidem, temazepam, cyclobenzaprine or carisoprodol.
